(19) United States
(12) Patent Application Publication
(10) Pub. No.: US 2025/0098962 A1
(43) Pub. Date: Mar. 27, 2025

(54) Soft Tissue Monitoring Device and Method

(71) Applicant: DOTPLOT LTD, Chislehurst Kent (GB)

(72) Inventors: Shefali Bohra, London (GB); Debra Babalola, London (GB); Yukun Ge, London (GB); Himari Tamamura, London (GB)

(21) Appl. No.: 18/832,692

(22) PCT Filed: Jan. 24, 2023
(86) PCT No.: PCT/GB2023/050154

§ 371 Date: Jul. 24, 2024

(30) Foreign Application Priority Data
Jan. 24, 2022 (GB) 2200891.6

(51) Int. Cl. A61B 5/00

(52) U.S. Cl. CPC A61B 5/0051; A61B 5/7267; A61B 5/744; A61B 2560/045; A61B 2560/0462; A61B 2562/0204; A61B 2562/0219; A61B 2562/0247

(57) ABSTRACT

Disclosed is a device, system and method for detecting and monitoring abnormalities in soft tissue based on acoustic signals transmitted through the soft tissue. The device includes a pressure sensor configured to sense pressure applied to a location of soft tissue by the device; and an acoustic generator configured to generate and emit an acoustic signal comprising a range of frequency components into the location of soft tissue when the pressure sensed by the pressure sensor exceeds a threshold pressure, an acoustic sensor configured to detect an acoustic signal produced from the interaction of the emitted acoustic signal with the soft tissue and to convert the detected signal to an audio data signal; and a transceiver configured to transmit the audio data signal to an external computing device. The system comprises the device and the external computing device. The external computing device is configured to receive the audio data signal from the device; and determine, using a machine learning model trained on a sample dataset of labelled audio data signals, a classification for the received audio data signal based on its frequency content indicating whether or not the respective location of soft tissue exhibits an abnormality.

### TECHNICAL FIELD OF THE INVENTION

The present application relates to a device, system and method for detecting and monitoring abnormalities (such as lumps) in soft tissue, particularly but not exclusively by self-examination.

### BACKGROUND OF THE INVENTION

Abnormalities in soft tissue can be benign or malignant. Malignant abnormalities in tissue may indicate the presence of a cancer and/or other long-term illnesses that develop close to or within the skin, e.g. breast cancer, testicular cancer, soft tissue sarcoma etc. Early detection of abnormalities in human or animal tissue is highly desirable, since early intervention can dramatically improve prognosis and recovery.

Palpation is a widely used technique to identify potentially problematic areas of soft tissue by feeling for lumps, bumps, size and texture inconsistencies of an organ or body part. In particular, breast cancer usually manifests as abnormalities such as lumps in breast tissue which can be detected through palpation. Palpation is a skilled technique usually performed by a healthcare practitioner as part of a routine check-up. However, women are advised to perform regular self-examinations of their breasts to keep track of any lumps or changes in their breasts to aid early lump detection. There is exists multiple techniques and methods for completing these self-checks, which can cause confusion and a lack of adherence to a regular self-examination routine, as well as lack in confidence that they are being performed correctly.

There exist a number of devices to aid examination of tissue and increase the likelihood of early lump detection. U.S. Pat. No. 8,006,319B2 discloses a device for breast self-examination which is worn over the fingers of a user's hand and configured to prevent non-recommended use of the thumb and palm during palpation and utilise a mineral oil to enhance sensitivity of touch and thereby aid palpation of breast tissue. As an alternative to palpation, iBreastExam™ is a portable, hand-held device developed for clinical use which uses capacitive sensing to measure tissue elasticity and provides real-time scan results that can be used to identify stiff tissue.

However, the devices and methods known in the art have limitations relating to useability, convenience, reliability of detection, and data access. For example, the palpation device in U.S. Pat. No. 8,006,319B2 still requires a level of experience and skill to correctly interpret what is felt, and the iBreastExam system is a clinical device that requires trained healthcare practitioners to use and analyse the data and is thus not suitable for regular self-examination. It is an aim of the present invention to overcome, or at least mitigate, deficiencies and drawbacks in the prior art.

### SUMMARY OF THE INVENTION

According to a first aspect of the invention, there is provided a device for detecting acoustic signals transmitted through soft tissue for use in detecting the presence of an abnormality in the soft tissue such as lumps in breasts or other areas of the body. The device may be a portable handheld device used for self-examination by the user. The device comprises a pressure sensor configured to sense pressure applied to a location of soft tissue by the device; an acoustic generator configured to generate and emit an acoustic signal into the location of soft tissue when the pressure sensed by the pressure sensor exceeds a threshold pressure; an acoustic sensor configured to detect an acoustic signal produced from the interaction of the emitted acoustic signal with the soft tissue; and a transceiver configured to transmit an audio data signal representing the detected acoustic signal to an external computing device.

In this context, the acoustic signal is a sound wave comprised of one or multiple or a range of different frequencies or frequency components. Preferably, the acoustic signal comprises a range of, or plurality of, frequency components and a predefined amplitude/intensity spectrum or spectral profile. Preferably, the acoustic signal comprises a range of frequencies in the audible range of up to 20 kHz. The acoustic generator may be configured to emit an acoustic signal comprising one or more or multiple frequencies within the range of 300 Hz to 19000 Hz, and preferably within a range of 600 Hz-6000 Hz, or any other suitable subrange.

Soft tissue act like a filter that attenuates certain frequencies or bands of frequencies more than others depending on the properties of the tissue such as density. As such, the detected acoustic signal has a modified amplitude spectrum that holds information that can be used, by the external computing device, to detect or determine the presence of an abnormality at the respective location of soft tissue. The acoustic measurement may be repeated at multiple different locations within an area of interest on the user's body, such as the torso, to produce scan data that can be used, by the external computing device, to build up a map of the scanned area indicating potentially problematic areas that need to be monitored or inspected/tested by a doctor. As such device may be referred to as a scan device. Scan data can be stored and compared to subsequent scan data to monitor the soft tissue and any identified abnormalities in the area of interest. Because the acoustic measurement is only performed once a predetermined threshold pressure is met, the scan data is reliable and reproducible.

The transceiver is preferably a wireless transceiver. The acoustic generator may be a speaker. The acoustic sensor is an acoustic transducer configured to convert the detected acoustic signal to an electronic audio data signal suitable for transmission. For example, the acoustic sensor may be a microphone.

The device may comprise a microcontroller configured to control operation of the device. The microcontroller may control the acoustic generator, acoustic sensor and transceiver. The microcontroller may be configured to control the transceiver to transmit the detected audio signal to an external computing device, e.g. in real time or after a time period, or at a certain time interval. The microprocessor may be configured to acquire and sample the audio data signal and optionally perform some on-board data pre-processing prior to transmission, such as filtering, averaging and/or smoothing.

The device may further comprise an inertial measurement unit configured to measure a position and orientation of the device (relative to the soft tissue). The transceiver may be configured to transmit position and orientation data to the external computing device along with the audio signal. In this way, each audio signal can be associated with a specific location of soft tissue.

The device may further comprise a rollerball mechanism configured to measure the speed and direction of the device as it is moved across a scan area of soft tissue. The roller ball mechanism comprises a ball configured to contact the skin and rotates as the device is moved across the scan area to provide one or more output signals that can be used to determine distance travelled, speed and direction of movement, e.g. similar to a mechanical computer mouse roller. The microcontroller may be configured to determine distance data from the one or more rollerball outputs. The microcontroller may be configured to control the transceiver to send or transmit, to the external computing device, distance data relating to the size of the area or part of the user's body.

Alternatively, the device may comprise a light emitting device and a photodetector for measuring the speed and direction of the device as it is moved across a scan area of soft tissue. The microcontroller may be configured to determine the speed and direction of the device as it is moved across a scan area of soft tissue based on the output of the photodetector, e.g. similar to a computer optical mouse.

The threshold pressure may be a location-specific or region-specific threshold pressure. The threshold pressure may be dependent on the measured position and orientation of the device relative to the soft tissue. The microcontroller may be configured to determine the desired threshold device based on the position and orientation data and an anatomical model, or a digital representation/model, of the scan area. Alternatively, the external computing device may determine the threshold pressure based on the received position data and an anatomical model, or a digital representation/model, of the scan area, and the threshold pressure may be set according to a control signal received from the external computing device.

The device may comprise a depth camera for generating two or three-dimensional image data of the area of interest. The external computing may be configured to use the image data to generate a customised anatomical model, or a digital representation/model, of the area of interest. The model may be a two or three-dimensional model. In addition to image date, the external computing may further be configured to use user body information, such as bra size and breast shape, provided by the user to generate the customised anatomical model, or a digital representation/model, of the area of interest.

The device may further comprise a handle. The handle may be retractable or collapsible for stowing the device.

The device may further comprise a battery. The battery may be a rechargeable battery. The device may comprise a charging coil for wirelessly charging the rechargeable battery. Alternatively, the device may comprise a power port for charging the rechargeable battery.

The wireless transceiver may be or comprise a Bluetooth transceiver module configured for wireless communication with the external computing device. The external computing device may be a mobile computing device such as a smart phone, tablet or laptop.

According to a second aspect of the invention, there is provided a system for detecting an abnormality in soft tissue. The system comprises the device of the first aspect and an external computing device in wireless communication with the device. The external computing device is configured to: receive an audio data signal from the device representing an acoustic signal detected from a location of soft tissue; and determine, using a machine learning model trained on a sample dataset of labelled audio data signals, a classification for the received audio data signal indicating whether or not the respective location of soft tissue exhibits an abnormality.

The classification is preferably based on analysis of the frequency content of the audio data signal. The audio data signal may be a time-domain signal. The external computing device may be configured to transform the audio data signal into amplitude/intensity data (frequency domain) comprising a plurality of frequency components. The external computing device may be configured to determine a sum of the intensity data across a plurality of predetermined frequency bands. The external computing device may be configured to calculate a first value and a second value using the summed intensity data, and classify the first and second values based on a comparison to classified/labelled first and second values in the sample dataset, wherein the classification of the coordinate indicates whether or not an abnormality is detected at the respective location of soft tissue. The external computing device may be configured to plot the first value and second value as coordinates; and classify the coordinates based on a comparison to classified coordinates in a sample dataset and/or classified clusters of coordinates in the sample dataset, wherein the classification of the coordinate indicates whether or not an abnormality is detected at the respective location of soft tissue.

The device may comprise an inertial measurement unit configured to measure a position and orientation of the device (relative to the soft tissue). The external computing device may further be configured to receive position data from the device and generate a spatially resolved map of the classification of respective locations of soft tissue as the device is moved across a scan area of soft tissue.

The map may be overlaid on an anatomical/digital model representing an area or part of the user's body that includes the scan area. The overlaid map may help guide the user to move the device to certain locations to complete a scan of the area.

The external computing device may be configured to generate a customised anatomical model of an area or part of the user's body that includes the scan area based on position and/or distance data (obtained from a roller ball or photodetector) received from the device. The external computing device may be configured to display the spatially resolved map over the anatomical model as the device is moved across the scan area of soft tissue.

The external computing device may comprise a graphical display.

The external computing device may be configured to generate a customised anatomical model of an area or part of the user's body based on two or three-dimensional image data of the area or part of the user's body. Optionally, the device comprises a camera configured to generate two or three dimensional image data, such as a depth camera.

The customised anatomical model may be a two or three-dimensional model. The anatomical model may be a torso model. The anatomical model may comprise a plurality of spatial segments representing a region on the body. The segments may be or comprise planar surfaces and/or polygons.

The external computing device may be configured to determine the position of the device on the user relative to the anatomical model (or customised anatomical model) based on the position and orientation data.

Where the customised anatomical model is a three-dimensional model, the external computing device may be configured to map the position of the device on the user onto the customised anatomical model based on the position and orientation (IMU) data and the respective normal vectors of segments of the customised anatomical model. The external computing device may be configured to convert the 3D customised anatomical model into a low polygon model (with reduced surfaces), determine the normal vector for each of the polygons in the model, and compare the position and orientation data to the normalised vectors to determine the device location. The external computing device may be configured to determine a normal vector of the orientation of the device with respect to a reference frame, and compare the normal vector of the device with the normal vectors from the 3D anatomical model to determine the device location. The location/polygon in the model corresponding to the closest matching normal vector may be identified as the position at which the device 100 is currently located on the user. In one implementation, the closest matching normal vector can be identified using k-dimensional spatial tree data structures.

The external computing device may be configured to determine a threshold pressure for triggering the emission of the acoustic signal by the device based on the received position/orientation data and an anatomical model (or the customised anatomical model) of an area or part of the user's body that includes the scan area. The external computing device may be configured to send the determined pressure threshold value to the device.

The anatomical model may contain threshold pressure information for each segment of the model. The external computing device may be configured to determine a threshold pressure for triggering the emission of the acoustic signal by the device based on the determined position of the device with respect to the anatomical model and threshold pressure information associated with that position/segment in the model. The external computing device may be configured to determine a pressure threshold value for detection of abnormalities for one or more regions of the anatomical model.

A position-dependent threshold pressure allows the variation in tissue density and/or skin/tissue thickness across the scan area to be taken into account, thereby providing more reliable, comparable and reproducible scan results. E.g. greater pressure is required where tissue thickness or skin thickness is greater and less pressure is required where the tissue thickness or skin thickness is lower. The threshold pressure may be approximately proportional to the tissue or skin thickness. The tissue thickness may be defined as the distance from the surface of the skin to the bone.

The device may comprise a rollerball mechanism or light emitter and photodetector system configured to measure the speed and direction of the device as it is moved across a scan area of soft tissue. The device may be configured to determine and send, to the external computing device, distance data relating to the size of the area or part of the user's body and position data relating to the position and orientation of the device on the area or part of the user's body. The external computing device may be configured to generate the customised baseline anatomical model for the user based on the received distance data and/or position data.

The external computing device may be configured to determine the position of the device on the user relative to the customised torso model based on the distance and/or position data. The external computing device may be configured to determine a pressure threshold value for detection of abnormalities for one or more regions of the customised anatomical model, and send the pressure threshold value to the device.

The area or part of the user's body may be or include the torso. The anatomical model may be or include a torso model or digital representation of the torso.

The external computing device may further be configured to determine a classification for an audio data signal obtained from a location on one of the user's breasts based on a comparison with an audio data signal obtained from a location on the other of the user's breasts.

The external computing device may further comprise a display, and wherein the external computing device is configured display the map on the display.

The classification may further indicate the depth of a detected tissue abnormality. The depth may be in the range of up to 15 mm, or up to 10 mm, or up to 8 mm.

The system may further comprise a charging unit for charging the device. Optionally or preferably, the device comprises a battery and a charging coil, the charging unit is a wireless charging unit. The charging unit may comprise a magnetic connector for securing the device to the charging unit.

According to a third aspect of the invention, there is provided a method for detecting an abnormality in soft tissue. The method comprises emitting, by a device, an acoustic signal into a location of soft tissue in response to a pressure exerted by the device on the location exceeding a threshold pressure; detecting, by the device, an acoustic signal produced from the interaction of the emitted acoustic signal with the soft tissue; converting the detected acoustic signal into audio data signal; and determining, using a machine learning model, a classification for the audio data signal indicating whether or not the respective location of soft tissue exhibits an abnormality. The machine learning model may be trained on a sample dataset of labelled audio data signals.

The acoustic signal may comprise a range of frequencies, preferably in the audible range. The step of emitting, by a device, an acoustic signal, may comprise emitting an acoustic signal comprising a range of frequencies within the range of 300 Hz-19000 Hz, and preferably within a range of 600 Hz-6000 Hz.

The classification is preferably based on analysis of the frequency content of the audio data signal. The audio data signal may be a time-domain signal. The method may comprise transforming the audio data signal into intensity data comprising a plurality of frequency components, and determining a sum of the intensity data across a plurality of predetermined frequency bands. The method may further comprise calculating a first value and a second value using the summed intensity data, and classifying the first and second values based on a comparison to classified/labelled first and second values in a sample dataset in the sample dataset, wherein the classification of the coordinate indicates whether or not an abnormality is detected at the respective location of soft tissue.

The method may comprise plotting the first value and second value as coordinates; and classifying the coordinates based on a comparison to classified coordinates in a sample dataset and/or classified clusters of coordinates in the sample dataset, wherein the classification of the coordinate indicates whether or not an abnormality is detected at the respective location of soft tissue.

The classification of the coordinates may further indicate the approximate depth of a detected tissue abnormality. The depth may be in the range of up to 15 mm, or up to 10 mm, or up to 8 mm. The classification may be no lump, a lump, a lump at 2 mm, a lump at 6 mm, a lump at 8 mm etc.

The location of soft tissue may be on the user's torso, and the method may further comprise identifying a quadrant of the torso in which the tissue abnormality is detected.

The method may comprise: measuring a position and/or orientation of a device configured to emit an acoustic signal into a location of soft tissue; determining a threshold pressure for triggering the emission of an acoustic signal by the device based on the measured position and orientation data and an anatomical model of an area or part of the user's body that includes the location; and emitting, by the device, an acoustic signal into a location of soft tissue in response to a pressure exerted by the device on the location exceeding the determined threshold pressure.

The method may comprise: determining the position of the device on the user relative to the anatomical model (or customised anatomical model) based on the position and orientation data. Where the customised anatomical model is a three-dimensional model, the method may comprise mapping the position of the device on the user onto the customised anatomical model based on the position and orientation (IMU) data and respective normal vectors of segments of the customised anatomical model. The method may comprise converting the 3D customised anatomical model into a low polygon model (with reduced surfaces), determining the normal vector for each of the polygons in the model, and comparing the position and orientation data to the normalised vectors to determine the device location. The method may comprise determining a normal vector of the orientation of the device with respect to a reference frame, and comparing the normal vector of the device with the normal vectors from the 3D anatomical model to determine the device location. The location/polygon in the model corresponding to the closest matching normal vector may be identified as the position at which the device is currently located on the user. The closest matching normal vector may be identified using k-dimensional spatial tree data structures.

The method may comprise determining a threshold pressure for triggering the emission of the acoustic signal by the device based on the received position/orientation data and an anatomical model (or the customised anatomical model) of an area or part of the user's body that includes the scan area, and sending the determined pressure threshold value to the device.

The anatomical model may contain threshold pressure information for each segment of the model. The method may comprise determining a threshold pressure for triggering the emission of the acoustic signal by the device based on the determined position of the device with respect to the anatomical model and threshold pressure information associated with that position/segment in the model. The method may comprise determining a pressure threshold value for detection of abnormalities for one or more regions of the anatomical model.

According to a fourth aspect of the invention, there is provided a method for processing audio data to detect an abnormality in soft tissue. The audio data may be time domain audio data representing sound that has travelled through soft tissue. The method is a computer-implemented method performed on a computing device.

The method may comprise receiving, from a device, time domain audio data, wherein the audio data represents sound that has travelled through soft tissue; transforming the audio data into intensity data comprising a plurality of frequency components; determining the sum of the intensity data within a plurality of predetermined frequency bands, calculating a first value and a second value using the summed intensity data, and classifying the first and second values based on a comparison to classified/labelled first and second values in a sample dataset, wherein the classification of the values indicates whether or not an abnormality is detected at the respective location of soft tissue.

The method may comprise plotting the first value and second value as coordinates; and classifying the coordinates based on a comparison to classified coordinates in a sample dataset and/or classified clusters of coordinates in the sample dataset, wherein the classification of the coordinate indicates whether or not an abnormality is detected at the respective location of soft tissue.

The method of the fourth aspect does not include a step of obtaining or measuring the time domain audio data (e.g. by the device).

The receiving step may be omitted. The method may comprise: (i) transforming a time domain audio data signal representing sound that has travelled through soft tissue into intensity data comprising a plurality of frequency components; (ii) determining the sum of the intensity data within a plurality of predetermined frequency bands; (iii) calculating a first value and a second value using the summed intensity data; and (iv) classifying the first and second values based on a comparison to classified/labelled values in a sample dataset, wherein the classification of the values indicates whether or not an abnormality is detected at the respective location of soft tissue. The method may comprise plotting the first value and second value as coordinates; and classifying the coordinates based on a comparison to classified/labelled coordinates in a sample dataset and/or classified clusters of coordinates in the sample dataset, wherein the classification of the coordinate indicates whether or not an abnormality is detected at the respective location of soft tissue.

The method may comprise providing (to a computing device) time domain audio data representing sound that has travelled through soft tissue. The method may comprise receiving, by the computing device from a device, time domain audio data representing sound that has travelled through soft tissue.

The classification of the coordinates may further indicate the depth of a detected tissue abnormality. The depth may be in the range of up to 15 mm, or up to 10 mm, or up to 8 mm.

The audio data signal may be associated with position data representing the location of soft tissue at which the audio data signal was acquired. The method may further comprise: repeating the processing steps (e.g.

steps (i) to (iv)) for a plurality of audio data signals obtained from/associated with a plurality of respective different locations of the soft tissue; and generating a spatially resolved map of the classification of the plurality of respective locations of soft tissue based on the position data.

The method may further comprise overlaying the map on an anatomical model representing an area or part of a body that includes the locations of soft tissue.

The device referred to in the above methods may be a device according to the first aspect.

According to a fifth aspect of the invention, there is provided a computer readable medium comprising executable instructions which, when executed by a processor or computing device, cause the processor or computing device to execute the method according to the third or fourth aspects.

Preferable features of the invention are defined in the appended dependent claims.

Features which are described in the context of separate aspects and embodiments of the invention may be used together and/or be interchangeable. Similarly, where features are, for brevity, described in the context of a single embodiment, these may also be provided separately or in any suitable sub-combination. Features described in connection with the device may have corresponding features definable with respect to the system and method(s), and vice versa, and these embodiments are specifically envisaged. Features described in connection with the system may have corresponding features definable with respect to the method(s), and vice versa, and these embodiments are specifically envisaged.

### BRIEF DESCRIPTION OF THE DRAWINGS

Embodiments of the invention will be described with reference to the figures in which:

FIGS. 1a-1f show views of a monitoring device according to an embodiment of the invention;

FIG. 2 is an exploded view of the device shown in FIGS. 1a-1f;

FIGS. 3a-3c show perspective views of a charging unit for a monitoring device according to an embodiment of the invention;

FIG. 4 is a schematic perspective view of a rollerball mechanism according to an embodiment of the invention;

FIGS. 5a-5d are charts showing relative audio intensity at different frequencies for no lump, 2 mm, 6 mm and 8 mm lump depth samples;

FIG. 6 is a plot of processed data samples for no lump and lumps at different depths;

FIG. 7 is a plot of actual lump classification against predicted lump classification; and

FIG. 8 is a flow diagram outlining the steps of lump detection according to an embodiment of the invention.

It should be noted that the figures are diagrammatic and may not be drawn to scale. Relative dimensions and proportions of parts of these figures may have been shown exaggerated or reduced in size, for the sake of clarity and convenience in the drawings. The same reference signs are generally used to refer to corresponding or similar features in modified and/or different embodiments.

### DETAILED DESCRIPTION

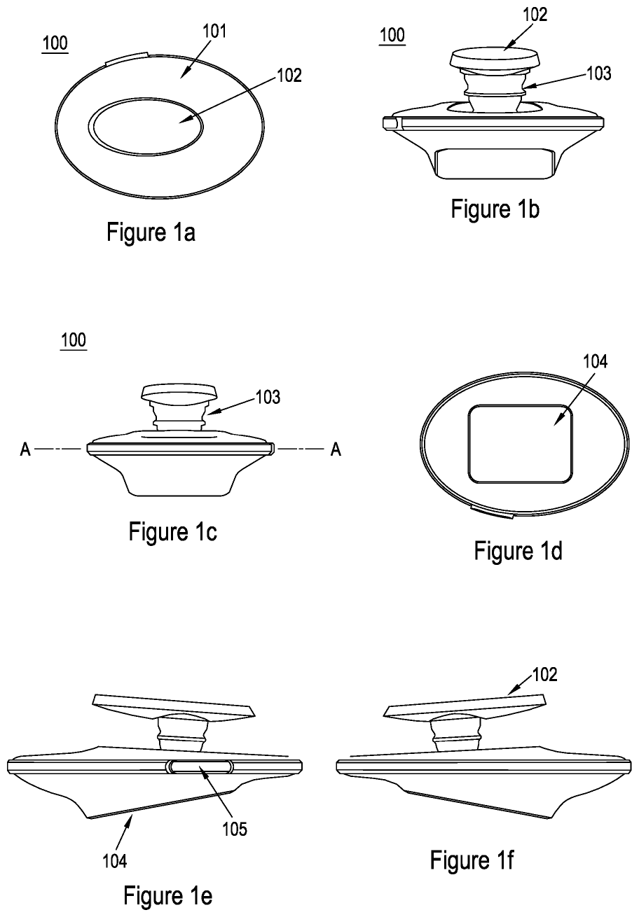

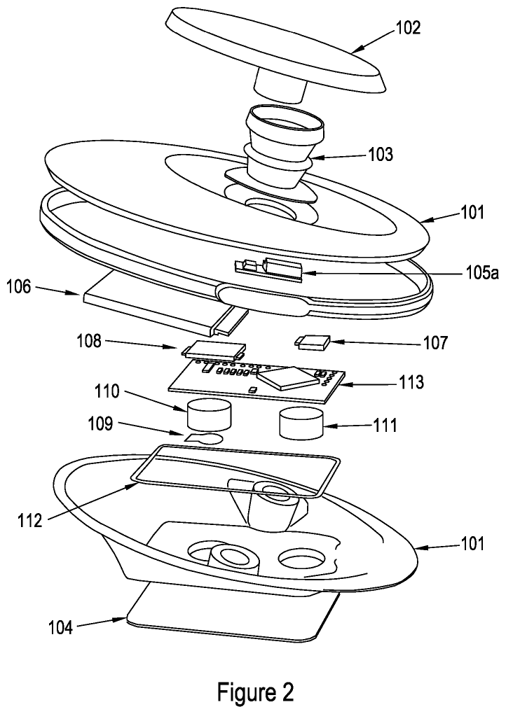

A device 100 for self-examination of tissue is shown in FIGS. 1a-1e and FIG. 2. Device 100 comprises body 101, handle 102 and stem 103. In use, stem 103 is held between, and gripped by, a user's fingers (typically between the index and middle fingers) to move and press surface 104 of body 101 against the skin. As can be seen in FIGS. 1e and 1f, surface 104 is substantially flat and lies at an angle relative to the plane A-A generally defined by body 101. Surface 104 is a soft fibre pad.

Handle 102 and the outer shell of body 101 may be manufactured from a durable material, such as a plastic, for example a polycarbonate. Stem 103 may be manufactured from a durable and flexible material such as elastomer (for example, silicone rubber) to allow a degree of relative movement between body 101 and handle 101. Stem 103 comprises one or more circumferential ridges and is cylindrically collapsible such that it can be folded within itself to allow handle portion 102 to be retracted and lie against, or at least closer to, oval portion 101 for safe and space-saving storage when device 100 is not in use.

On/off switch portion 105 is located on the side of body 101 and is at least partially transparent to allow light from an LED to be visible as an indicator of when device 100 is powered and is in use or is ready for use.

Some internal components of device 100 are shown in FIG. 2. Lithium battery 106 provides power to device components. Electronic switch 105a comprises an LED which is activated when switch 105a is ‘on’. A microcontroller on PCB 113 controls acoustic generator (speaker) 110, acoustic sensor (microphone) 111, pressure sensor 109, charging coil 112, internal measurement unit (IMU) 108 and transceiver 107.

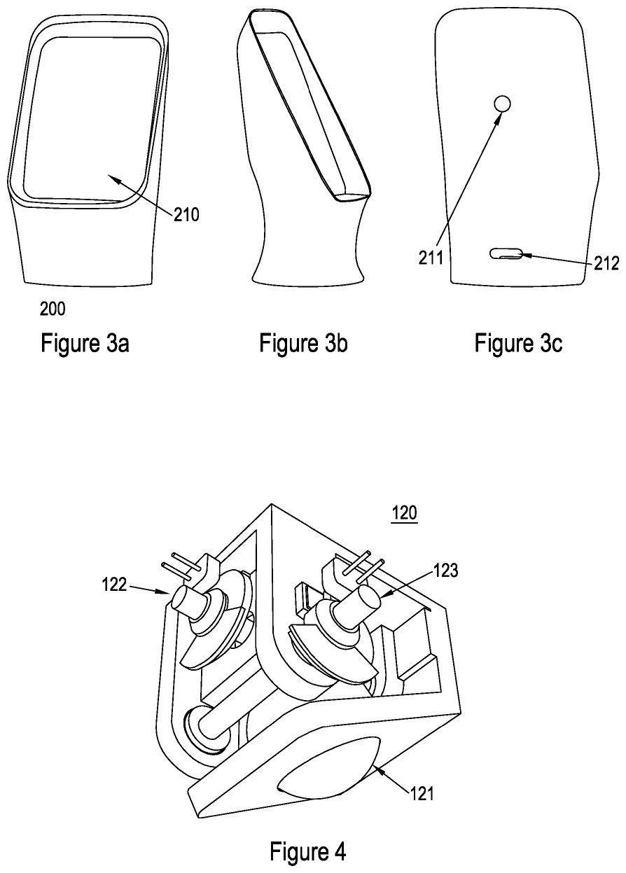

Wireless charging coil 112 facilitates charging of device 100 using a charging unit as shown in FIGS. 3a, 3b and 3c. Device 100 preferably also comprises a rollerball mechanism (not shown in FIGS. 1a-1f or FIG. 2) for measuring distance moved by device 100 across a torso.

Transceiver 107 is preferably a short-range transceiver module for wireless communication, such as a Bluetooth transceiver module. Transceiver 107 is configured to wirelessly receive and transmit data with an external computing device, such as a smartphone.

Pressure sensor 109 is preferably a high precision sensor such as thin film pressure sensor RP-C7.6-LT. Pressure sensor 109 measures the pressure applied to soft tissue by device 100 as a user presses device 100 (specifically, surface 104) against the skin. When the measured pressure reaches a predetermined threshold value, generator 110 generates (under instruction from microcontroller 113) an acoustic signal which is emitted for a predetermined time period, for example, 1 second. The frequency of the sound emitted by generator 110 has multiple frequencies which are preferably within the range 300 Hz-19000 Hz, and further preferably within the range 600 Hz-6000 Hz. Using frequencies below the ultrasound range reduces the cost of the generator component and advantageously improves useability for the user since the emitted sound is audible to a user and thereby informs the user of when the threshold pressure has been reached and that the reading has been taken.

Sensor 111 is preferably an electret condenser microphone which receives sound emitted by generator 110 after the sound has been reflected by the body. As the sound travels through the body, its frequency may change as a result of changes in soft tissue density. Such density changes may be incremental, but analysis of the change in frequency of the sound detected over time may indicate the presence of developing tissue abnormalities. A data acquisition device such as a national instruments NI USE-6211 is used to convert the received sound as audio data. The sampling frequency is preferably between 10,000-250,000 samples per second. The captured audio data is periodically transmitted wirelessly by transceiver 107 to an external computing device, which processes the audio data, as described below. The data may be sent to the external device in near real-time—i.e. immediately after each time the generator emits sound. Alternatively, device 100 may further comprise storage for storing the audio data collected in respect of each time the generator emits sound during a process of self-examination of tissue using device 100.

FIGS. 3a, 3b and 3c show charging unit 200 for device 100. Charging unit 200 comprises connector 212 for receiving a power cable. Charging unit 200 further includes wireless communication unit 211 which facilitates communication with device 100 so as to allow device 100 to be located if prompted by device 100, and vice versa. In one embodiment, charging unit 200 comprises a basic user interface feature which instructs charging unit 200 to wirelessly communicate with device 100 and instruct device 100 to emit an audible noise, e.g. should the device 100 go missing. Surface 210 is adapted to receive surface 104 so as to support device 100. Charging unit 200 also comprises a magnetic connector (not shown) beneath surface 210 which secures surface 104 of device 100 to surface 210.

In order to process the audio data to detect tissue abnormality, a baseline model of tissue density variation across a torso can be used. The size and shape of the breasts and torso area is different for each user. It was found by the present inventors that use of a customised model for each user provided more accurate abnormality detection results. The customised torso or anatomical model is a digital representation of the area of the body and requires dimensions of the user's torso to scale the model. These can be obtained from optic measurements, e.g. from two or three dimensional image data obtained from a depth camera or LiDAR, or by physical measurements obtained from a measuring device.

In an embodiment, device 100 also comprises roller unit 120 to obtain physical measurements of the torso dimensions, as shown in FIG. 4. Roller unit 120 has a similar structure to the ball mechanism of a computer mouse and comprises ball 121, x-roller 123 and y-roller 122. Ball 121 drives the x-roller and y-roller to move together when rolled across the surface of a user's torso. Following calibration, the speed and angle, and distance travelled of the x-roller and y-roller can be calculated (e.g. distance travelled can be based on the number of revolutions considering the roller ball's circumference), which in turn allows the position of roller unit 120 on the torso to be determined and the distance moved by roller unit 120 can be determined. This distance information can be obtained by the user moving device 100 between the user's left collarbone and left nipple, between the user's right collarbone and right nipple and between the left and right nipple, and preferably also from left underarm to right underarm. This distance information is used to determine the scale by which to resize a baseline torso model. The baseline torso model may be retrieved from an external computing system and the customised model may be stored both locally on the user's device and/or at the external computing system.

In a preferred embodiment, device 100 is used in conjunction with a software application running on an external computing device (not shown), such as a mobile computing device (smart phone, tablet, laptop etc.). The external computing device and device 100 communicate wirelessly. Due to computational capacity and storage limitations, the external computing device may delegate some or all data processing to a separate entity, such as a cloud server, which is in communication with the external computing device.

In a preferred embodiment, the external computing device comprises an infrared (IR) depth camera module (e.g. an IR dot matrix projector and IR camera) and is configured to generate a three-dimensional model of the user's torso based on the output of the depth camera. This 3D model is used to resize a baseline torso model and produce the customised torso model. In one implementation, where the external computing is a smart phone such as an iPhone, the face ID system can be used.

A software application running on the external computing device may prompt a user to conduct a tissue examination periodically. For example, a user may be prompted by a mobile application running on their smartphone/mobile device to self-examine their breasts every month. The user's customised torso model is generated on the first self-examination. The customisation of the torso is further enhanced by receiving bra size and breast shape information from the user. Subsequent self-examinations use the customised torso model to collect audio data. The customised map may or may not require recalibration every few months (preferably 2-6 months) in order to account for changes to the body (e.g., weight gain, pregnancy, menopause etc.).

The area of the user's customised torso is divided into regions or segments. For each region, the user may be guided by the mobile application on the external computing device to apply pressure to the breast tissue as they hold device 100 to enable device 100 to obtain audio data. As the user conducts a self-examination and moves device 100 to different torso regions, an image or anatomical model of the user's torso may be displayed by the software application and the user's progress in completing the required application of device 100 in each region is visibly indicated, e.g. by overlaying the map. Alternatively, the user may use the device 100 without guidance from the software application.

IMU 108 (6-axis or 9-axis) comprises a gyroscope and an accelerometer, and a magnetometer. The orientation and position of device 100 relative to a global reference frame is obtained by IMU 108 and sent to the external computing device to allow the position of device 100 to be mapped on to the customised torso model as the user moves device 100 to different regions of the torso and applies sufficient pressure to activate acoustic generator 110. Since the breast area is not planar, the orientation measurement provides useful information, in conjunction with position relative to a global reference frame as to where in the torso region device 100 is located. The device 100 with IMU can be calibrated by placing it on a flat area of the chest to set a reference plane for understanding the direction in which a user is standing (in reference to the gravitational field of earth) while making a scan. The device 100 may instead use an attitude and heading reference system (AHRS) to provide position and orientation information, as is known the art.

In one implementation, the external computing device is configured to map the position of the device 100 onto the customised torso model using the position and orientation (IMU) data by computing the normal vectors of each segment of the torso model and comparing the position and orientation data to the normalised vectors. Specifically, the 3D customised torso model is processed and converted into a low polygon model with reduced surfaces, and the normal vector for each of the polygons in the model is determined. While the device 100 is in each position on the user's torso, the rotation of the IMU 108 with respect to the reference frame is calculated and the normal vector of the orientation of the device 100 at that position is calculated. For example, depending on how the IMU 108 is oriented in the device 100, the normal vector of the device 100 may be the +z axis (0,0,1) when there is no rotation, and the rotated normal vector is calculated by multiplying the quaternion from the IMU 108 with +z axis. The rotated normal vector obtained is then compared with the normal vectors from the 3D torso model to look for the closest match. The location/polygon corresponding to the closest matching normal vector is identified as the position at which the device 100 is currently located. In one implementation, the closest matching normal vector can be identified using k-dimensional spatial tree data structures.

The accuracy of the position determined using position/orientation data from IMU 108 is increased by using IMU data collected from other users (using separate devices) as a training dataset for a machine learning model. The trained model can then identify, to a higher accuracy than use of the specific IMU data in isolation, which region of the breast device 100 is currently located in based on the specific IMU data for a particular device. This can be achieved by using the k-nearest neighbours (KNN) algorithm. When the user places device 100 at a certain position on the chest/torso, IMU 108 records angle values of device 100 and corresponding XYZ position data. This position data is compared (using, for example, MATLAB's NumNeighbors function) with position data at different, known torso locations in the model/database to derive specific location information. For example, X, Y and Z axes for the device are recorded as A, B, and C respectively. We compare this data with the ABC data for different chest locations in the database to derive its location information. The data in the database/model is a 3D plot, and the X-axis, Y-axis, and Z-axis are A, B, and C, respectively. In one example, NumNeighbors=10 or 1 can be used when comparing.

For each region or segment of a torso, the pressure threshold necessary to activate generator 110 may differ. This is because of the variation in tissue density across the breast area, which necessitates varying pressure in order to identify any density changes which may indicate an abnormality. The threshold pressure for each region is adjusted for a particular user using the customised torso map. Greater pressure is required where the skin/tissue is thicker and less pressure is required where the skin/tissue is thinner. The threshold pressure is approximately proportional to the thickness of the skin or tissue (where skin/tissue thickness is defined as the distance from the surface of the skin to the bone). In one example, threshold pressure information for a given location or region of a torso is associated respective locations in the torso model. The external computing device can then determine the correct threshold pressure values for the device 100 to use based on the determined position of the device and the threshold pressure information at that location/region in the torso model, and send the threshold values to the device 100 so that the audio data is acquired at the correct pressure. The threshold pressure information is stored on the external computing device along with the torso model. The threshold pressure required may alternatively be determined by measuring the average pressure applied to each torso segment during clinical palpation.

The audio data collected by device 100 for each region of the customised torso is processed separately to determine whether the breast tissue within a region or section contains an abnormality. The processed data for each region and relating to each month's self-examination is stored and compared with data from future examinations to identify changes in the breast tissue in a region or regions. Any identified changes in the density of breast tissue in a particular region can be reviewed.

Breast tissue acts as a frequency filter for frequencies lower than ultrasound. The captured audio data is therefore used to distinguish between a normal and abnormal tissue by comparing the audio data with previous readings for a particular user, as well as comparing it against a baseline index. The baseline index is based on a sample dataset, which comprises frequency changes caused by lumps at predefined depths. This baseline dataset was used to train a machine learning model.

To generate the baseline dataset, device 100 was used on a material having varying density at different depths to simulate lumps at different depths in breast tissue. The material used was Ecoflex™ 00-30, although other materials may be suitable.

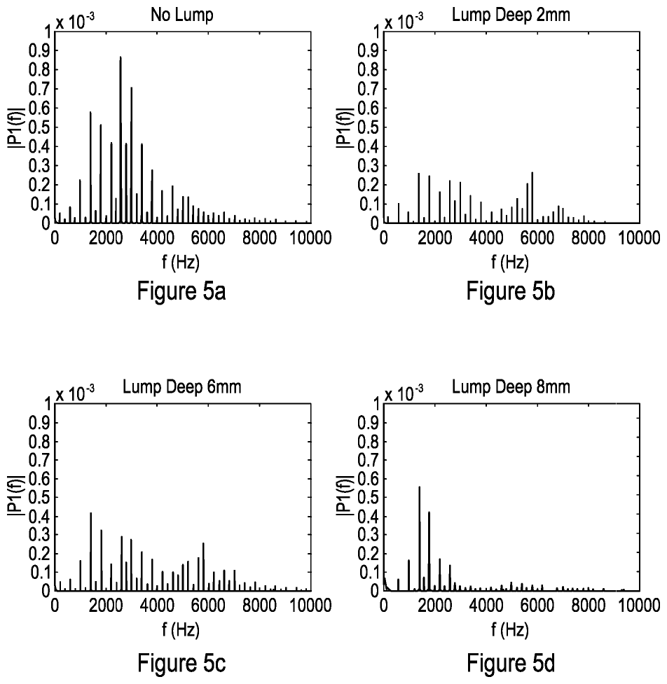

Using device 100, audio data was collected when the device was used on the simulated soft tissue having lumps at depths of 2 mm, 4 mm, 6 mm and 8 mm. The lumps were 3D-printed using TPU. Each lump depth was sampled multiple times. The audio data captured for each sample represents captured sound having a range of frequencies. For each sample, the audio data undergoes Fourier transformation (using, for example, MATLAB) to audio intensity. FIGS. 5a-5b show the intensity values of the audio data for samples where there was no lump, a lump at 2 mm, a lump at 6 mm and a lump at 8 mm respectively. It was found that frequencies within the range of 50 Hz to 8000 Hz underwent the most noticeable variation at different lump depths.

The Fourier transformation splits the audio data for each sample into at least two bands—for example, 0 Hz to 2000 Hz, 2000 Hz to 4000 Hz and 4000 Hz to 8000 hz. The sum of the intensity across each band is determined and the sum value is denoted by values A, B and C respectively. A is divided by B and B is divided by C and the values of A/B and B/C are input to a k-nearest neighbours (KNN) algorithm which compares the A/B and B/C values to earlier values of A/B and B/C.

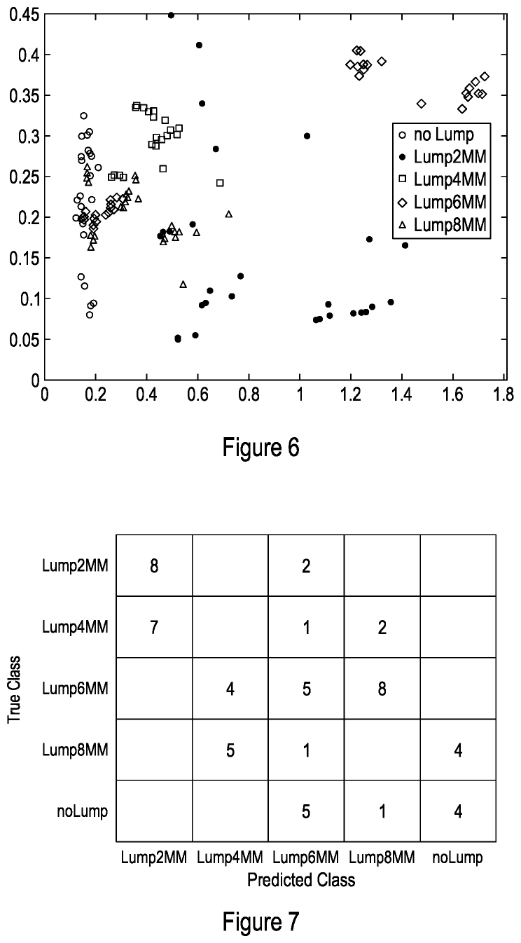

The values of A/B and B/C are plotted as shown in FIG. 6a, wherein the y axis is A/B and the x axis is B/C. As can be seen, the data for lumps of different depths occupy different regions on the plot, but data for lumps of a particular depth lie close together to form one or more clusters. This is the baseline dataset. A k-Nearest Neighbor (KNN) is therefore used to classify a data sample into one of the clusters, and thereby a conclusion—i.e. ‘no lump’ or a lump at a depth of 2, 4, 6, or 8 mm—can be extrapolated. FIG. 6b compares lump classification predicted by a KNN algorithm with the actual classification of a lump. Based on the present data, the accuracy of correctly predicting lump depth is 40%, but the accuracy of correctly predicting whether or not a lump is present is 90%.

An alternative way of using KNN for sound classification would be to compare the value of A/B/C to earlier values of A/B/C in the same way as above.

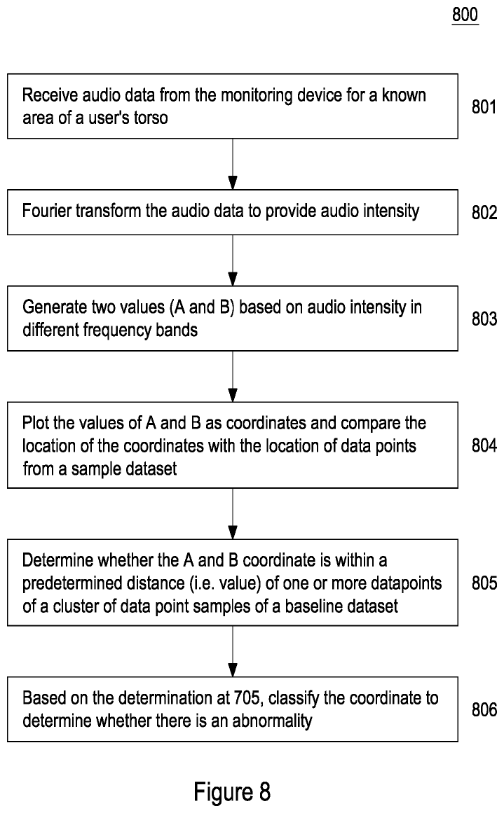

Processing and classification of audio data received from device 100 is described with reference to FIG. 8. After the audio data from device 100 is received by the user's mobile computing device (step 801), the audio data may be sent to a processing system such as a cloud server for processing and for inclusion in training data for one or more machine learning models. The audio data may also be stored locally on the user's mobile device for processing. In various embodiments, the device 100 and/or the external computing device is camera-free for improved privacy.

The received audio data from a user's device is processed similarly to the samples used to create the baseline dataset, as described above. Accordingly, the user's audio data for each sample collected in respect of each region of the user torso undergoes Fourier transformation into intensity (step 802), and values of A/B and B/C for each sample are calculated (step 803) and are plotted (step 804) similarly to FIG. 6a. Each datapoint is analysed (using MATLAB's NumNeighbors parameter, for example, which may be set to 1) (step 805) to determine which cluster the data point is closest to, and to then conclude, for each torso segment, whether there is no lump, or a lump at a 2, 4, 6, or 8 mm (step 806). Although not shown, the inventor's results indicate that the device 100 can be used to detect lumps at depths of up to 15 mm. It will be appreciated that while the audio data must be acquired at some point, the above classification method is a method of processing the audio data, and does not include the physical step of data collection. For example, the audio data could be acquired at some time in the past or by a different device.

After each self-examination (i.e. after the user has used device 100 across the complete torso area and all necessary data has been collected for each torso segment/region), the application running on the mobile device presents the lump detection determination results as a 2D map. This map is then compared with previous maps to identify any change in the lump detection results (for example, a 10% or 20% difference may indicate the development of an abnormality). Finally, the quadrant of the breast in which an abnormality is detected is identified. The user can choose to share this data with a clinician to increase the efficiency of further investigation by a clinician.

From reading the present disclosure, other variations and modifications will be apparent to the skilled person. Such variations and modifications may involve equivalent and other features which are already known in the art, and which may be used instead of, or in addition to, features already described herein.

Although the appended claims are directed to particular combinations of features, it should be understood that the scope of the disclosure of the present invention also includes any novel feature or any novel combination of features disclosed herein either explicitly or implicitly or any generalisation thereof, whether or not it relates to the same invention as presently claimed in any claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable sub-combination.

1. A device for detecting acoustic signals transmitted through soft tissue, comprising
a pressure sensor configured to sense pressure applied to a location of soft tissue by the device;
an acoustic generator configured to generate and emit an acoustic signal into the location of soft tissue when the pressure sensed by the pressure sensor exceeds a threshold pressure, wherein the acoustic signal comprises a range of frequency components and a predefined amplitude spectrum;
an acoustic sensor configured to detect an acoustic signal with a modified amplitude spectrum produced from the interaction of the emitted acoustic signal with the soft tissue and to convert the detected signal to an audio data signal;
a transceiver configured to transmit the audio data signal to an external computing device; and
a microcontroller configured to control operation of the acoustic generator, acoustic sensor and transceiver, wherein the microcontroller is further configured to control the transceiver to transmit the audio signal to an external computing device.

2. The device of claim 1, further comprising an inertial measurement unit configured to measure a position and orientation of the device, and wherein the transceiver is configured to transmit position and orientation data to the external computing device.

3. The device of claim 1, further comprising a rollerball mechanism configured to measure the speed and direction of the device as it is moved across a scan area of soft tissue, and wherein the device is configured to determine and send, to the external computing device, distance data relating to the size of the area or part of the user's body; or wherein the acoustic generator is configured to emit an acoustic signal comprising frequencies within the range of 30 0Hz-19000 Hz, and preferably within a range of 600 Hz-6000 Hz.

4. (canceled)

5. The device of claim 2, wherein the threshold pressure is dependent on the measured position and orientation of the device relative to the soft tissue, and optionally, wherein the threshold pressure is set according to a control signal received from the external computing device.

6. A system for detecting an abnormality in soft tissue, comprising the device of claim 1 and an external computing device in wireless communication with the device, wherein the external computing device is configured to:
receive an audio data signal from the device representing an acoustic signal detected from a location of soft tissue; and
determine, using a machine learning model trained on a sample dataset of labelled audio data signals, a classification for the received audio data signal based on its frequency content indicating whether or not the respective location of soft tissue exhibits an abnormality.

7. The system of claim 6, wherein the audio data signal is a time-domain signal and the external computing device is configured to:
transform the audio data signal into intensity data comprising a plurality of frequency components;
determine a sum of the intensity data across a plurality of predetermined frequency bands,
calculate a first value and a second value using the summed intensity data and plot the first value and second value as coordinates; and
classify the coordinates based on a comparison to classified coordinates in a sample dataset and/or classified clusters of coordinates in the sample dataset, wherein the classification of the coordinate indicates whether or not an abnormality is detected at the respective location of soft tissue.

8. The system of claim 7, wherein the device comprises an inertial measurement unit configured to measure a position and orientation of the device relative to the soft tissue, and wherein the external computing device is further configured to receive position data from the device and generate a spatially resolved map of the classification of respective locations of soft tissue as the device is moved across a scan area of soft tissue.

9. The system of claim 8, wherein the map is overlaid on an anatomical model representing an area or part of the user's body that includes the scan area; or, wherein the external computing device is configured to generate a customised anatomical model of an area or part of the user's body that includes the scan area based on position data received from the device and a baseline anatomical model of the area, and display the spatially resolved map over the customised anatomical model as the device is moved across the scan area of soft tissue; and optionally or preferably, wherein generating the customised anatomical model comprises rescaling the baseline model based on the position data; or, wherein the external computing device is configured to generate a customised anatomical model of an area or part of the user's body based on two or three dimensional image data of the area or part of the user's body and a baseline anatomical model of the area, and display the spatially resolved map over the customised anatomical model as the device is moved across the scan area of soft tissue; and optionally or preferably, wherein generating the customised anatomical model comprises rescaling the baseline model based on the image data; and further optionally or preferably, wherein the device comprises a camera configured to generate two or three dimensional image data, such as a depth camera; or, wherein the external computing device is further configured to:
determine a threshold pressure value for triggering the emission of the acoustic signal by the device based on the received position data and an anatomical model of an area or part of the user's body that includes the scan area;
and send the threshold pressure value to the device.

10. (canceled)

11. (canceled)

12. (canceled)

13. The system of claim 9, wherein the device comprises a rollerball mechanism configured to measure the speed and direction of the device as it is moved across a scan area of soft tissue,
wherein the device is configured to determine and send, to the external computing device, distance data relating to the size of the area or part of the user's body and position data relating to the position and orientation of the device on the area or part of the user's body;
wherein the external computing device is configured to:
generate the customised anatomical model for the user based on the received distance data;
determine the position of the device on the user relative to the customised anatomical model based on the distance and/or position data; and
determine a pressure threshold value for detection of abnormalities for one or more regions of the customised anatomical model, and send the pressure threshold value to the device.

14. The system of claim 9, wherein the external computing device is configured to:
generate a three-dimensional customised anatomical model for the user based on three-dimensional image data of the area or part of the user's body;
map the position of the device on the user to a region in the customised torso model based on the received position data and surface normal vectors of regions in the customised anatomical model;
determine a pressure threshold value for detection of abnormalities at the device position based on information associated with the mapped region in the customised anatomical model; and
send the pressure threshold value to the device.

15. The system of claim 9, wherein the area or part of the user's body is or includes the torso, and the anatomical model is or includes a torso model.

16. The system of claim 15, wherein the external computing device is further configured to determine a classification for an audio data signal obtained from a location on one of the user's breasts based on a comparison with an audio data signal obtained from a location on the other of the user's breasts.

17. The system of claim 9, wherein the external computing device further comprises a display, and wherein the external computing device is configured to display the map on the display; or wherein the classification further indicates the depth of a detected tissue abnormality.

18. (canceled)

19. A method for detecting an abnormality in soft tissue, comprising
emitting, by a device, an acoustic signal into a location of soft tissue in response to a pressure exerted by the device on the location exceeding a threshold pressure, wherein the acoustic signal comprises a range of frequencies;
detecting, by the device, an acoustic signal produced from the interaction of the emitted acoustic signal with the soft tissue;
converting the detected acoustic signal into audio data signal; and
determining, using a machine learning model trained on a sample dataset of labelled audio data signals, a classification for the audio data signal based on its frequency content indicating whether or not the respective location of soft tissue exhibits an abnormality.

20. The method of claim 19, wherein the audio data signal is a time-domain signal and the method comprises:
transforming the audio data signal into intensity data comprising a plurality of frequency components;
determining a sum of the intensity data across a plurality of predetermined frequency bands,
calculating a first value and a second value using the summed intensity data and plot the first value and second value as coordinates; and
classifying the coordinates based on a comparison to classified coordinates in a sample dataset and/or classified clusters of coordinates in the sample dataset, wherein the classification of the coordinate indicates whether or not an abnormality is detected at the respective location of soft tissue.

21. The method of claim 19, wherein the classification of the coordinates further indicates the depth of a detected tissue abnormality; or wherein the step of emitting, by a device, an acoustic signal, comprises emitting an acoustic signal comprising a range of frequencies within the range of 300 Hz-19000 Hz, and preferably within a range of 600 Hz-6000 Hz; or wherein the location of soft tissue is on the user's torso, and the method further comprises identifying a quadrant of the torso in which the tissue abnormality is detected.

22. (canceled)

23. (canceled)

24. The method of claim 20, further comprising:
measuring a position and orientation of a device configured to emit an acoustic signal into a location of soft tissue;
determining a threshold pressure for triggering the emission of an acoustic signal by the device based on the measured position and orientation data and an anatomical model of an area or part of the user's body that includes the location; and
emitting, by the device, an acoustic signal into a location of soft tissue in response to a pressure exerted by the device on the location exceeding the determined threshold pressure; and optionally, wherein the step of determining a threshold pressure comprises:
generating a three-dimensional customised anatomical model for the user based on three-dimensional image data of the area or part of the user's body;
mapping the position of the device on the user to a region in the customised torso model based on the received position data and surface normal vectors of the customised anatomical model;
determining a pressure threshold value for detection of abnormalities at the device position based on information associated with the mapped region in the customised anatomical model; and
sending the pressure threshold value to the device.

25. (canceled)

26. A method of processing time domain audio data representing sound that has travelled through soft tissue to detect an abnormality in the soft tissue, comprising:
(i) transforming a time domain audio data signal into intensity data comprising a plurality of frequency components;
(ii) determining the sum of the intensity data within a plurality of predetermined frequency bands,
(iii) calculating a first value and a second value using the summed intensity data and plot the first value and second value as coordinates; and
(iv) classifying the coordinates based on a comparison to classified coordinates in a sample dataset and/or classified clusters of coordinates in a sample dataset, wherein the classification of the coordinate indicates whether or not an abnormality is detected at the respective location of soft tissue.

27. The method of claim 26, wherein the classification of the coordinates further indicates the depth of a detected tissue abnormality.

28. The method of claim 26 or 27, wherein the audio data signal is associated with position data representing the location of soft tissue at which the audio data signal was acquired, and the method further comprises:
repeating steps (i) to (iv) for a plurality of audio data signals obtained from a plurality of respective different locations of the soft tissue; and
generating a spatially resolved map of the classification of the plurality of respective locations of soft tissue based on the position data; and optionally, further comprising overlaying the map on an anatomical model representing an area or part of a body that includes the locations of soft tissue.

29. (canceled)
